(19) United States
(12) Patent Application Publication
(10) Pub. No.: US 2019/0102818 A1
(43) Pub. Date: Apr. 4, 2019

(54) EFFICIENT PATIENT DISCHARGE AND COMMUNICATION SYSTEM FOR MEDICAL EQUIPMENT

(71) Applicant: MEDELA HOLDING AG, Baar (CH)

(72) Inventors: Alan Cawley, McHenry, IL (US); Kevin Ackermann, McHenry, IL (US); Michael Lauletta, McHenry, IL (US)

(21) Appl. No.: 16/034,184

(22) Filed: Jul. 12, 2018

Related U.S. Application Data
(60) Provisional application No. 62/568,179, filed on Oct. 4, 2017.

(51) Int. Cl. G06Q 30/06; G16H 40/20; G16H 40/40; G06Q 10/08

(52) U.S. Cl. CPC G06Q 30/0613; G16H 40/20; G16H 10/60; G06Q 10/0833; G16H 40/40

(57) ABSTRACT

An efficient patient discharge system is set forth. The system includes a MAPIT device configured to generate a MAPIT request from an orderer and deliver the MAPIT request to at least one eligible provider device. The eligible provider device is configured to selectively generate and send responsive signals to the MAPIT request, based on selection criteria, via the MAPIT device, including delivery information. The selection criteria for connecting to the MAPIT device being stored in a processor operatively connected to a selection circuit of the MAPIT device. The at least one eligible provider device is enabled by the selection circuit to generate and send updates to the processor.

### CROSS-REFERENCE TO RELATED APPLICATIONS

This patent application claims priority to U.S. Provisional Patent Application No. 62/568,179 filed Oct. 4, 2017, entitled “Efficient Patient Discharge And Communication System For Medical Equipment,” the entire disclosure of which is hereby incorporated by reference.

### FIELD OF THE DISCLOSURE

The present disclosure relates generally to a system that facilitates efficient provisioning between a manufacturer, provider, such as an eligible durable medical equipment provider (DME), and at least one ordering facility (orderer), such as a doctor's office or hospital discharge system, and further enables efficient confirmation, ordering and delivery of medical supplies from providers to medical and residential settings.

### BACKGROUND

Medical equipment providers, such as DME's, can deliver products from a variety of medical device manufacturers to medical and/or home facilities for patients. Manufacturers can also provide direct delivery services of their products. Certain providers, such as DME's, can have great flexibility in coordinating bundles of requested equipment to fill a particular order from a number of different manufacturers.

Some systems are known that allow for differentiation as to which items, products or services are directed to a consumer and which require a local provider, as discussed, for example, in US Patent Application No. 2002/0007290. The system includes a distributed electronic cataloging, order taking, submission, shipping and payment system interacting under direction of an established plan formulary. Historical data related to the subscribing entity is reviewed as a prelude to preparation of one or more plan formularies which the subscribing entity wishes to automate. A Provider Network Manager assigns the appropriate local provider. If the order is declined, the PNM can select another local provider who can deliver the order. The local provider either accepts or declines the order.

Such systems incorporate personal information including medical and payment information, and require both a complicated plan and the PNM to interact with the system in order to facilitate processing of an order, and can become inefficient where the PNM selects a provider who declines the order request. Further, manufacturers cannot improve the availability of their products or optimize an order based on feedback related to previous orders fulfilled by a provider. As a result, there exists a need to streamline placement and fulfillment of orders for medical device equipment that reduces complexity of the known systems, while improving the efficiency, scope and reliability of the ordering process.

### SUMMARY

In one exemplary embodiment of an efficient patient discharge system, the system includes a MAPIT device configured to generate a MAPIT request from an orderer and deliver the MAPIT request to at least one eligible provider device. The eligible provider device is configured to selectively generate and send responsive signals to the MAPIT request, based on selection criteria, via the MAPIT device, including delivery information. The selection criteria for connecting to the MAPIT device being stored in a processor operatively connected to a selection circuit of the MAPIT device. The at least one eligible provider device is enabled by the selection circuit to generate and send updates to the processor.

In another exemplary embodiment, the system includes a drainage pump device configured for both hospital and post discharge use. The drainage pump device can include a suitable location detector or location enabling software operatively connected, either directly or indirectly, to the MAPIT device. The location detector can enable transfer of the device information from the hospital to the post discharge location. The location detector can be configured to enable the processor to generate and transmit an error message within the system if transfer is attempted before the drainage pump device ownership change information is confirmed by the MAPIT device.

In yet another embodiment, the drainage pump device is further defined by a wound therapy device.

In still another embodiment, the drainage pump device is further defined by at least one of a breastmilk extraction device, a chest drainage device and an enteral feeding device.

In another exemplary embodiment, the system can include a selection circuit having selection criteria imported in a bulk file to the processor from the at least one provider device.

In other embodiments, the at least one provider device of the system can be configured to provide real time responses and signals to the MAPIT device.

In still another embodiment, the MAPIT device of the system can be configured to generate and send supply orders to the at least one provider device based on usage information sent from the drainage pump device to the MAPIT device.

In yet another system constructed in accordance with the principles herein, the MAPIT device can be configured to generate and send supply orders to the at least one provider device based on usage information sent from the drainage pump device to the MAPIT device.

An exemplary method of transferring a drainage pump device from hospital to a post care facility, constructed in accordance with the principles herein, can include at least the following steps: providing a device suitable for use in both the hospital and a post care facility; connecting the device to an efficient patient discharge system; notifying the discharge system if the device location changes where a change of ownership confirmation information is not received by the system and processed by a processor of the system; and generating an error message regarding the location of the device.

The exemplary method can further include operatively connecting a memory storage to the processor, the memory storage including recordable instructions executed by a component of the system.

Various advantages of the present disclosure are specifically described below in reference to the exemplary embodiments, or conceptually embodied therein, and all possible combinations of the drawings and claims are contemplated hereby. The drawings and description herein are provided to merely illustrate examples of the general concepts discussed throughout the present disclosure. Numerous changes and modifications can be made, as known to those of skill in the art, without departing from the general principles set forth herein. In addition, all patents and publications referenced are incorporated herein by reference in the entirety.

### BRIEF DESCRIPTION OF THE DRAWINGS

These and other features and advantages of the various exemplary embodiments disclosed herein will be better understood with respect to the following description and drawings, in which:

FIG. 1 is an embodiment of a known patient discharge system;

FIG. 2 is an exemplary embodiment of an efficient patient discharge system;

FIG. 3 is an exemplary embodiment of another efficient patient discharge system, where the system can prioritize eligible providers based on feedback received indirectly or directly through the system from a patient, orderer, manufacturer, administrator, or post care facility;

FIG. 4 is an exemplary embodiment of an efficient patient discharge system configured to process new order and supply requests to the manufacturer from the Provider either automatically or based on an inventory confirmation from the Provider in the system;

FIG. 5 is a flow chart of an exemplary method of the present disclosure; and

FIG. 6 is an exemplary embodiment configured to receive location information from a suitable component of the system, such as a suitable drainage pump device.

Common reference numerals are used throughout the drawings and the detailed description to indicate the same elements.

### DETAILED DESCRIPTION

The detailed description set forth below in connection with the appended drawings is intended as a description of certain exemplary embodiments of various system components constructed in accordance with the principles herein. These examples are not intended to represent the only embodiments or forms that may be developed or utilized according to these principles.

Certain aspects of some embodiments constructed in accordance with the principles herein are directed toward an efficient patient discharge system including an eligible provider feedback and prioritizing system that allows a manufacturer to customize the parameters chosen to select and continually update eligible provider's list(s) within the system based on system feedback. Certain aspects of some embodiments constructed in accordance with the principles herein include efficient ordering capabilities from a provider to a manufacturer via the system and can be based on information stored and updated according to use of the system. Other aspects of some embodiments constructed in accordance with the principles herein include an efficient patient discharge system that provides an electronic provider request from an orderer directly to one or more providers, and enables the first eligible provider that responds to the electronic request to proceed to fill the order. Each provider can use their own internal mechanisms to determine patient eligibility and manage payment.

Unlike the know systems, the manufacturer is connected to the system, but the manufacturer is not necessarily contacted unless there is a system event requiring intervention by the manufacturer. For example, in some embodiments, in the event of a delay or problem with any request the system can notify the manufacturer/administrator so the manufacturer can intervene and unencumber the process. In other embodiments, the manufacturer can be contacted to confirm an order update from the provider so that the provider can proceed to accept an order request.

Other embodiments are contemplated, wherein system information input by the administrator, provider, orderer, care facility or patient is sorted and correlated to prioritize the information based on manufacturer settings in the system, thereby generating the top eligible providers at any given time that will provide the most efficient patient discharge system experience as determined by the manufacturer. The timing of the generating step can occur when an orderer selects an order request or MAPIT button, or can occur on a scheduled update time, or in real time (continuously) based on other sorting parameters input by the manufacturer.

As will be described in more detail below, several embodiments are contemplated in accordance with the principles herein, some of which are shown in the exemplary embodiments.

It is understood that the systems described herein may be used to select a provider to deliver a wide range of products from multiple medical equipment manufacturers. Such products will be collectively referred to herein as “bundles” for purposes of simplicity. The system can be connected to each manufacturer, and the collective manufacturer parameters can be mathematically averaged to determine the parameters for an eligible provider for the system based on the items listed in the bundles identified in the order. Further, the provider can select a portion of items from the list of items in the bundles of the order, and the remaining items can continue to display as a revised bundles order request that other eligible providers can accept. In this manner, highly efficient bundles and sub bundles the orders can occur via the efficient patient discharge system.

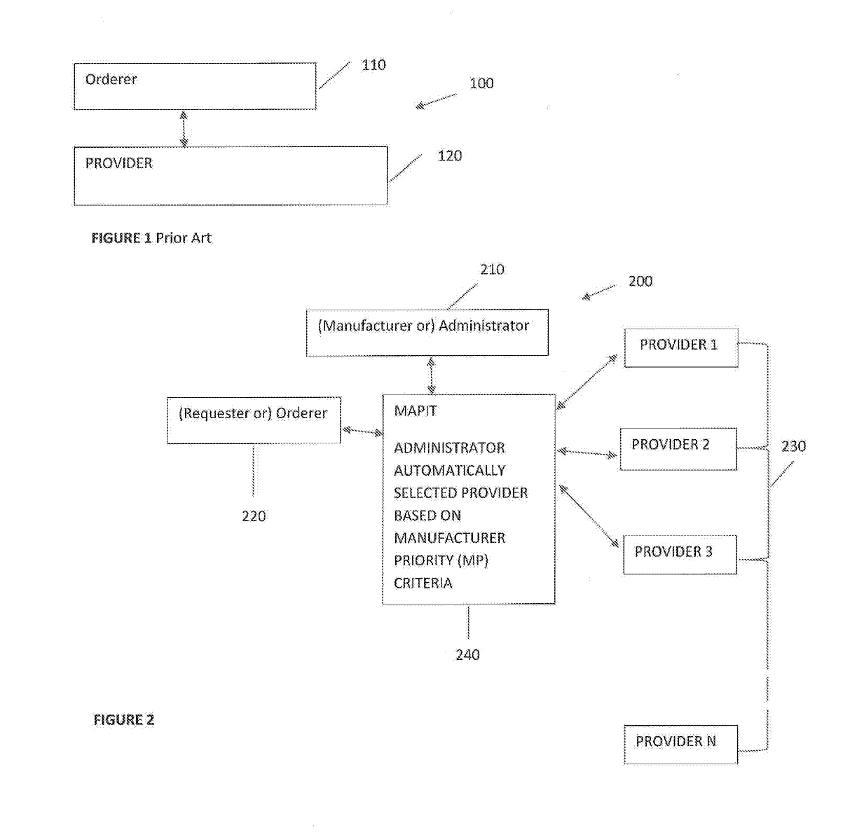

As illustrated in FIG. 1, some known patient discharge systems shown generally at 100 involve a discharge coordinator contacting a provider, such as a DME, to present information in the discharge order and to coordinate getting the order filled via the contact. A number of inefficiencies could occur in this system, such as waiting to reach the provider or waiting to the provider to investigate whether they can take an order based on any of a number of conditions of the transaction, including whether the patient's insurance type is accepted. The provider might also offer alternate devices based on inventory, or be unable to support transfer of a device from one facility to another.

As illustrated in FIG. 2, an efficient patient discharge system constructed in accordance with the principles herein, shown generally at 200, can include an Administrator 210, such as one or more manufacturers, an orderer 220, and at least one provider 230 connected to the system. The system can include a MAPIT button or command that allows the orderer to enter an order request that is sent to eligible providers 230 automatically based on manufacturer priority (MP) criteria. Although the Administrator 210 is connected to the system, the Administrator only becomes engaged in the transaction in the event that a problem or delay arises in the process. eligible providers 230 can include a group of providers'. The 1^{st} eligible provider to accept the MAPIT request gets the order or the portion of the order accepted. The provider can set or modify delivery area parameters in response to a request, and can display the delivery area information on a map in the system for the orderer to view. In this manner, the orderer can see the capabilities of each provider who accepts a MAPIT request, and can gain information about the capabilities of the provider.

In an embodiment, the system can be configured to match eligible providers based on a variety of parameters, for example location derived from the MAPIT request, patient information, zip code input by orderer, ability to accept health insurance coverage, or based on information contained in a patient's electronic medical record (EMR). If desired, the system can learn the zip code information and generate separate eligible provider lists based on zip code sorting, each such list then undergoing system updates according to manufacturer parameters and according to a selected schedule. Alternatively, the system can generate regional lists based on zip code information or past order acceptance information.

As illustrated in FIG. 3, an efficient patient discharge system shown generally at 300 can include one or more information pathways that can send feedback information into the MAPIT program processor from a variety of sources such as a patient 320, orderer 330, provider 340 post care facility 350, or administrator 360, for example. The administrator, or manufacturer, can determine how to sort and correlate the feedback information to optimize the efficiency of the system with continual collection of feedback and updates to the list of eligible providers.

An advantage of the system is that the administrator can quickly intervene once it is notified that a problem exists with filling the order. The Administrator can then take next steps quickly to overcome any inefficiencies associated with a particular order. The system thus provides one or more manufacturers an opportunity to intervene and assist when needed to support providers and improve the delivery of services to the patient.

Additionally, the system allows an administrator to confirm a transfer of existing devices for the patient to all relevant parties in seconds with real time updates. Thus, the system increases the confidence that device therapy started in a hospital will continue with the patient in the post care environment.

In an embodiment, the system software can attach the MAPIT request to a patient ID. The system can transfer insurance information to one or more eligible providers to transfer or fill a MAPIT request.

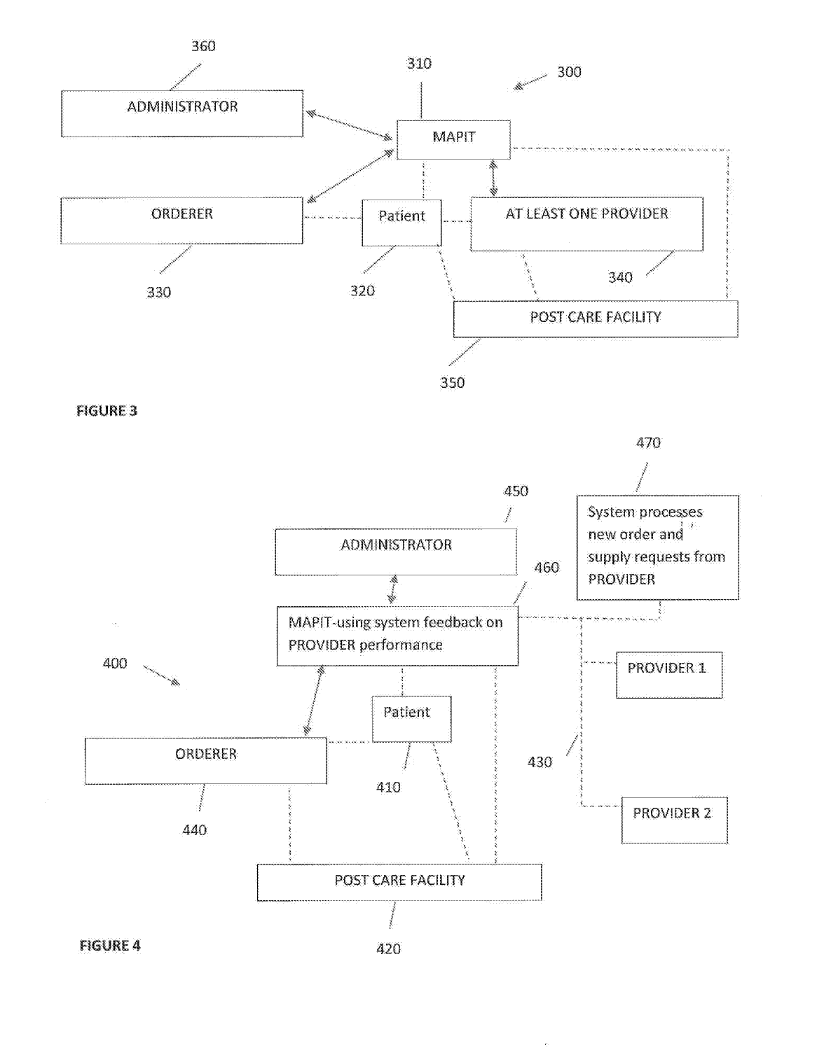

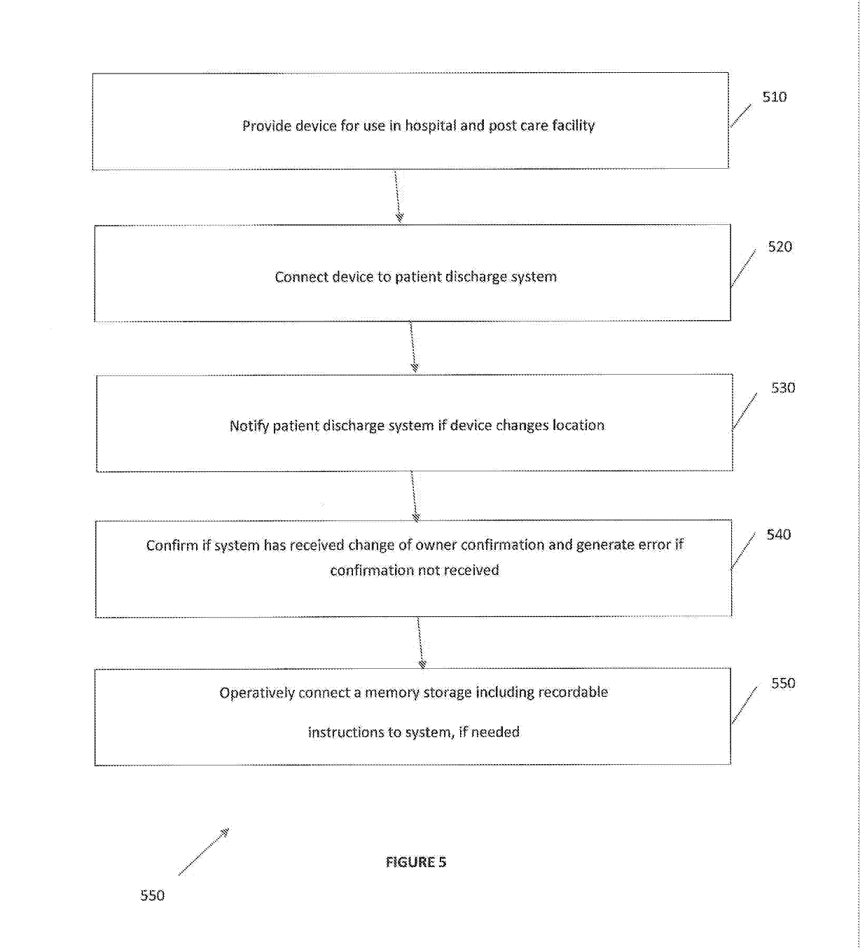

In the exemplary embodiment illustrated in FIG. 4, an efficient patient discharge system is shown generally at 400. The system 400 can allow a patient 410 or others to provide feedback regarding a number of details of the device and usage including device issues, delivery experience, or to present questions or ask for help. The patient can interact through a care provider at a post care facility 420 or through a provider 430.

In an embodiment, final delivery for the order is time stamped and/or confirmed with an orderer 440. In an embodiment, a provider confirms delivery, and delivery can be set according to an estimated delivery schedule. Order entry, acceptance, or delivery information can be generated in a notice that includes both the orderer 440 and an Administrator 450 via MAPIT 460, if desired. The Administrator 450 can intervene to improve the delivery outcome, if desired.

In an embodiment, ongoing status of the device and any renewal information are both available to the Administrator 450 and to the patient 410 through MAPIT.

In all embodiments, any field or data set of the system can be gathered and analyzed to extract information for a manufacturer or to rate provider eligibility. Preferably data set(s) exclude patient identifying information. Provider 430 eligibility ratings can be incorporated into the system 400 at the MAPIT 460 system processor, based on performance and/or patient 410 satisfaction or any other suitable parameter.

The system 400 can store and update information concerning a provider's supply or device inventory, and automatically generate a reorder notification to the Administrator 450, where the updates can be based on MAPIT orders accepted, if desired, or on a number of inputs selected by the provider 430. Alternatively, the system 400 can process new orders for supplies or devices based on a supply request 470 generated by a provider via the system 400. In some embodiments, the system can automatically generate a confirmation of inventory to the must be confirmed by a provider before the MAPIT request can be accepted. In this manner, orders will not be awarded to providers that fail to confirm inventory availability in response to the system generated inventory confirmation request.

In an embodiment, a patient or provider can discontinue therapy and document why therapy was discontinued. The Discontinue Therapy information can be routed back to the orderer to confirm via the system 400.

A system constructed in accordance with the principles herein can notify one or more Administrators in real time of delivery plans so that they can intervene if needed in an order to improve the efficiency of the system. The system can thus automate and reduce patient discharge time. Further, in all embodiments, any field or data set of the system can be gathered and analyzed to extract information for the manufacturer or to rate provider eligibility. Preferably, data set(s) exclude patient identifying information. Provider eligibility ratings can be based on a number of factors, and can include performance and/or patient satisfaction.

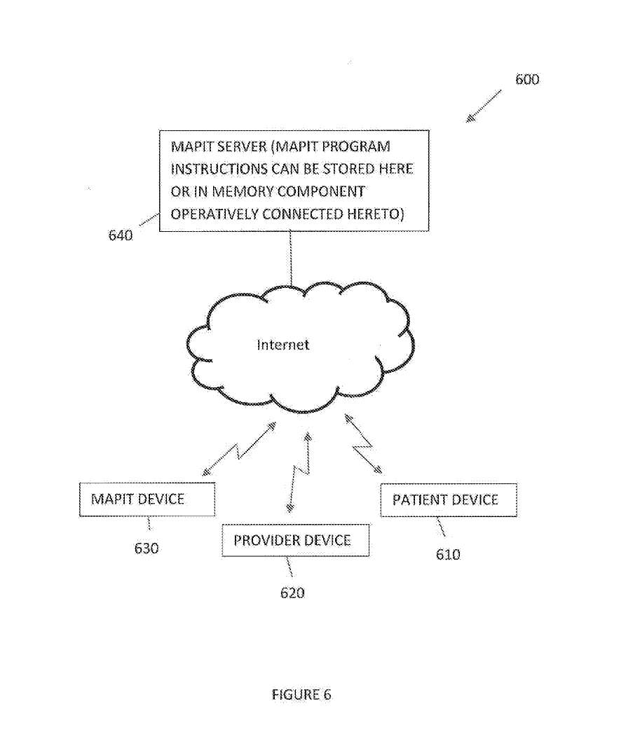

FIG. 5 is a flow chart of a method, shown generally at 500, according to the system disclosed herein. The exemplary method of transferring a drainage pump device from hospital to a post care facility, constructed in accordance with the principles herein, can include at least the following steps: providing a device suitable for use in both the hospital and a post care facility at 510; connecting the device to an efficient patient discharge system at 520; notifying the discharge system if the device location changes where a change of ownership confirmation information is not received by the system and processed by a processor of the system at 530; and generating an error message in the system regarding the location of the device at 540. The exemplary method can further include the step of operatively connecting a memory storage to the processor, the memory storage including recordable instructions executed by a component of the system at 550.

FIG. 6 is an exemplary embodiment of a system shown generally at 600 configured to receive location information from a suitable component of the system, such as a suitable drainage pump device 610. Examples of suitable drainage pump devices can include, for example, at least one of a breastmilk extraction device, a chest drainage device and an enteral feeding device or any other suitable device. Alternatively, the system can connect to additional or other suitable components that are intended to transfer from hospital to a post discharge care location 610, where a change in ownership information is generated by the system during the patient discharge process. A provider device 620 can be added to the discharge system at any time, and selectively operatively connected or linked to the MAPIT device 630 based on satisfying selection criteria of the system. A MAPIT server 640 can provide an associated memory component and/or processor, operatively connected thereto, for storing MAPIT program instructions, carrying out instructions of the program, and generating responsive signals to input from the devices 610, 620, 630.

In some further embodiments, any of the foregoing systems and methods of the present disclosure and exemplary aspects may alternatively and/or additionally utilize blockchain technology. For example, a blockchain may be used in transferring a drainage pump device from the hospital to a post care facility, such as in identifying the drainage pump device based upon a request from the orderer. In addition, a blockchain may be used in rating a provider based upon data received from one or more of the patient, the orderer, the provider, the post care facility or the manufacturer. Further, a blockchain may be used in monitoring and updating data relative to one or more of the drainage pump device, the patient, the orderer, the provider, the post care facility, or the manufacturer. In one example, the systems and methods described herein allow for using a blockchain of one or more of the patient discharge system, the hospital, the post care facility, the provider, and/or the manufacturer. In some examples, the blockchain may be a private blockchain, which gives the option for private information, such as private patient data, and permissioned participants in the blockchain.

More specifically, and in one example, the method of transferring a drainage pump device from a hospital to a post care facility may further comprise identifying, via one or more processors, a blockchain associated with one or more of the patient discharge system, the hospital, and the post care facility. The method may then further comprise accessing, at a memory or memory storage coupled to one or more processors, the blockchain corresponding to one or more of the patient discharge system, the hospital, and the post care facility to retrieve information about the drainage pump device, such as the device location.

In another example, the method may further comprise receiving, at the processor, information about the drainage pump device, and updating, at the memory, a block of a blockchain of one or more of the patient discharge system, the hospital, the post care facility, or the manufacturer to include and/or indicate information about the drainage pump device received.

In yet another example, the method may further comprise: receiving, at the processor, information about a provider of the drainage pump device; calculating, via one or more processors, a rating of the provider based upon information received about the provider; and updating, at the memory or memory storage, a block of a blockchain of one or more of the patient discharge system, the hospital, the post care facility, or the manufacturer to include and/or indicate information about the provider, including the information received and the rating calculated.

Variations of the specific system configurations shown and described herein that provide an efficient connection between discharge orderer and 1^{st} available eligible provider, while incorporating options such as notice, feedback and ordering information to an administrator, such as a manufacturer, are within the scope of the principles of the present disclosure, and are included in all claims deriving therefrom.

What is claimed:

1. An efficient patient discharge system comprising:
a MAPIT device;
the MAPIT device configured to generate a MAPIT request from an orderer and deliver the MAPIT request via the MAPIT device to at least one eligible provider device;
the at least one eligible provider device configured to selectively generate and send responsive signals to the MAPIT request, based on selection criteria, via the MAPIT device including delivery information;
the selection criteria for connecting to the MAPIT device being stored in a processor operatively connected to a selection circuit of the MAPIT device;
the at least one eligible provider device enabled by the selection circuit to generate and send updates to the processor.

2. The system of claim 1, further comprising;
a drainage pump device configured for both hospital and post discharge use, the drainage pump device further comprising a location detector operatively connected to the MAPIT device, the location detector enabling transfer of the device information from the hospital to the post discharge location, the location detector causing the processor to generate and transmit an error message if transfer is attempted before the drainage pump device ownership change information is confirmed by the MAPIT device.

3. The system of claim 1, the drainage pump device further defined by a wound therapy device.

4. The system of claim 1, the drainage pump device further defined by at least one of a breastmilk extraction device, a chest drainage device and an enteral feeding device.

5. The system of claim 1, the selection circuit including selection criteria imported in a bulk file to the processor from the at least one provider device.

6. The system of claim 1, the at least one provider device configured to provide real time responses and signals to the MAPIT device.

7. The system of claim 3, the MAPIT device configured to generate and send supply orders to the at least one provider device based on usage information sent from the drainage pump device to the MAPIT device.

8. The system of claim 4, the MAPIT device configured to generate and send supply orders to the at least one provider device based on usage information sent from the drainage pump device to the MAPIT device.

9. A method of transferring a drainage pump device from hospital to a post care facility comprising the following steps:
providing a device suitable for use in both the hospital and a post care facility;
connecting the device to the efficient patient discharge system of claim 1;
notifying the discharge system if the device location changes where a change of ownership confirmation is not received by the system and processed by a processor of the system; and
generating an error message regarding the location of the device.

10. A method as claimed in claim 9 further comprising:
a memory storage operatively connected to the processor, the memory storage including recordable instructions executed by a component of the system.

11. A method as claimed in claim 9, further comprising:
identifying, via one or more processors, a blockchain associated with one or more of the patient discharge system, the hospital, and the post care facility;
accessing, at a memory coupled to one or more processors, the blockchain corresponding to one or more of the patient discharge system, the hospital, and the post care facility to retrieve information about the drainage pump device, such as the device location.

12. A method as claimed in claim 9, further comprising receiving, at the processor, information about the drainage pump device, and updating, at the memory, a block of a blockchain of one or more of the patient discharge system, the hospital, the post care facility, or the manufacturer to include and/or indicate information about the drainage pump device received.

13. A method as claimed in claim 9, further comprising: receiving, at the processor, information about a provider of the drainage pump device; calculating, via one or more processors, a rating of the provider based upon information received about the provider, and updating, at the memory, a block of a blockchain of one or more of the patient discharge system, the hospital, the post care facility, or the manufacturer to include and/or indicate information about the provider, including information received and the rating calculated.

14. The system of claim 1, further comprising a blockchain, and the at least one eligible provider device adapted to update, at a memory coupled to the processor, a block of the blockchain.
